(19) United States
(12) Patent Application Publication
(10) Pub. No.: US 2014/0093469 A1
(43) Pub. Date: Apr. 3, 2014

(54) SHINE-PRODUCING HAIR-STYLING PRODUCT WITH A STRONG HOLD, PROVIDING HAIR WITH A PLEASANT FEEL

(71) Applicant: Henkel AG & Co. KGaA, Duesseldorf (DE)

(72) Inventors: Burkhard Mueller, Duesseldorf (DE); Susanne Schmarje, Hamburg (DE)

(73) Assignee: Henkel AG & Co., KGaA, Duesseldorf (DE)

(21) Appl. No.: 14/100,125

(22) Filed: Dec. 9, 2013

Related U.S. Application Data
(63) Continuation of application No. PCT/EP2012/058887, filed on May 14, 2012.

(30) Foreign Application Priority Data
Jun. 10, 2011 (DE) 10 2011 077 373.8

(51) Int. Cl. A61K 8/81; A61Q 5/12; A61Q 5/06

(52) U.S. Cl. CPC A61K 8/8152; A61K 8/8117; A61K 8/8147; A61Q 5/06; A61Q 5/12
USPC 424/70.16

(57) ABSTRACT

Agents for treating keratin-containing fibers, in particular human hair, containing, in a cosmetically acceptable carrier: at least one anionic setting copolymer encompassing at least one structural unit of formula (I) and at least one structural unit of formula (II) and at least one structural unit of formula (III), in which R¹ denotes a hydrogen atom or a methyl group, R² denotes a hydrogen atom or a methyl group, R³ denotes a branched (C₁ to C₂₀) alkyl group, a branched (C₂ to C₆) hydroxyalkyl group, or a *-(CH₂CH₂O)ₙ—R⁶ group where R⁶=branched (C₁ to C₂₀) alkyl and n=1 to 30, R⁴ and R⁵ denote a hydrogen atom, or one of the two residues denotes a hydrogen atom and the other a methyl group, are suitable for temporary reshaping of hair and for imparting shine, in particular as an aerosol hair spray or aerosol hair foam.

### TECHNICAL FIELD

The present invention generally relates to temporary reshaping of keratin-containing fibers, in particular of human hair, and more particularly relates to agents for hair treatment containing at least one special anionic copolymer derived from vinyl aromatic compounds and esters of acrylic acid or methacrylic acid with branched alcohols, to the use of said agents for the temporary reshaping and/or care of keratin-containing fibers, and to aerosol sprays or aerosol foams based on said agents.

### BACKGROUND

The initially most important property of an agent for temporary reshaping of keratinic fibers, hereinafter also called a “styling agent,” is to give the strongest possible hold to the treated fibers in the shape that has been generated. In addition to a high degree of hold, styling agents must also meet a large number of further requirements. These can be subdivided roughly into: properties on the hair; properties of the particular formulation, e.g. properties of the foam, gel, or sprayed aerosol; and properties that relate to the handling of the styling agent, the properties on the hair being of particular importance. Moisture resistance, low tack, and a balanced conditioning effect may be mentioned in particular. In addition, a styling agent should be universally usable for, if possible, all types of hair.

A plurality of synthetic polymers that are utilized in styling agents have already been developed in order to meet the various requirements. The polymers can be subdivided into cationic, anionic, nonionic, and amphoteric film-forming and/or setting polymers. Ideally, upon application to the hair in even a small quantity, the polymers yield a polymer film that on the one hand imparts a strong hold to the hairstyle but on the other hand is sufficiently flexible not to break under stress. If the polymer is too brittle, this results in the formation of so-called “film plaques,” i.e. residues that detach as the hair moves and give the impression that the user of the corresponding styling agent has dandruff. In addition, hair coated with a setting polymer often feels rough.

In addition to the strong hairstyle hold, the temporarily styled hair should moreover look healthy and natural. Hair shine plays a dominant role here. So-called shine agents are therefore often added in sufficient quantity to the hair styling agents. These shine agents are, for example, oils or shine-imparting pigments such as, for example, mica particles. Shine-imparting particles have the disadvantage that they detach from the hair over time and collect again after some time on, for example, clothing or facial skin. Oils make the hair heavy and in some cases result in degraded adhesion of the film-forming or setting polymers on the hair. This possibly leads to the disadvantage that the constructed hairstyle cannot be immobilized for a sufficient period of time by the film-forming or setting polymers. The hairstyle collapses more quickly.

The object of the present invention was therefore to make available an agent for the temporary reshaping and/or care of keratinic fibers that is notable for a high degree of hold, for outstanding shine on the keratin-containing fibers, and for good sensory characteristics of the keratin-containing fibers (in particular, a soft hair feel).

It has now been found, surprisingly, that this can be achieved by way of at least one special anionic copolymer derived from vinyl aromatic compounds and esters of acrylic acid or methacrylic acid with branched alcohols.

Furthermore, other desirable features and characteristics of the present invention will become apparent from the subsequent detailed description and the appended claims, taken in conjunction with this background.

### BRIEF SUMMARY

In an embodiment, an agent for treating keratin-containing fibers, comprises, in a cosmetically acceptable carrier: at least one anionic setting copolymer encompassing at least one structural unit of formula (I) and at least one structural unit of formula (II) and at least one structural unit of formula (III)

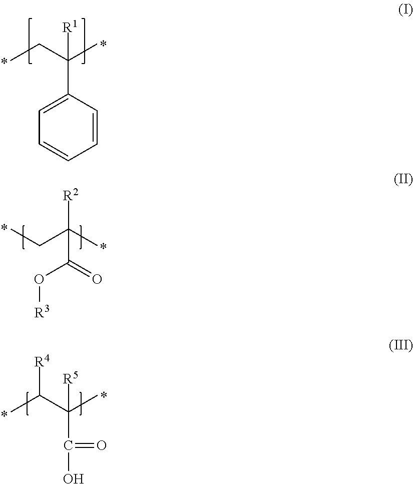

in which R¹ denotes a hydrogen atom or a methyl group, R² denotes a hydrogen atom or a methyl group, R³ denotes a branched (C₁ to C₂₀) alkyl group, a branched (C₂ to C₆) hydroxyalkyl group, or a *-(CH₂CH₂O)ₙ—R⁶ group where R⁶=branched (C₁ to C₂₀) alkyl and n=1 to 30, and R⁴ and R⁵ denote a hydrogen atom, or one of the two residues denotes a hydrogen atom and the other a methyl group.

In an embodiment, a kit of parts comprises an aerosol delivery apparatus and a composition comprising, based on its weight:

30 to 80 wt % of an agent for treating keratin-containing fibers, comprising, in a cosmetically acceptable carrier: at least one anionic setting copolymer comprising at least one structural unit of formula (I) and at least one structural unit of formula (II) and at least one structural unit of formula (III)

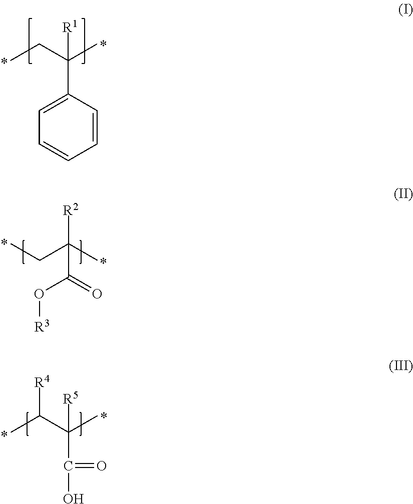

in which
R¹ denotes a hydrogen atom or a methyl group, R² denotes a hydrogen atom or methyl group, R³ denotes a branched (C₁ to C₂₀) alkyl group, a branched (C₂ to C₆) hydroxyalkyl group, or a *-(CH₂CH₂O)ₙ—R⁶ group where R⁶=branched (C₁ to C₂₀) alkyl and n=1 to 30, R⁴ and R⁵ denote a hydrogen atom, or one of the two residues denotes a hydrogen atom and the other a methyl group, and

20 to 70 wt % of at least one propellant.

In an embodiment, a method for treating keratin-containing fibers comprises, applying an agent for treating keratin-containing fibers, comprising, in a cosmetically acceptable carrier: at least one anionic setting copolymer comprising at least one structural unit of formula (I) and at least one structural unit of formula (II) and at least one structural unit of formula (III)

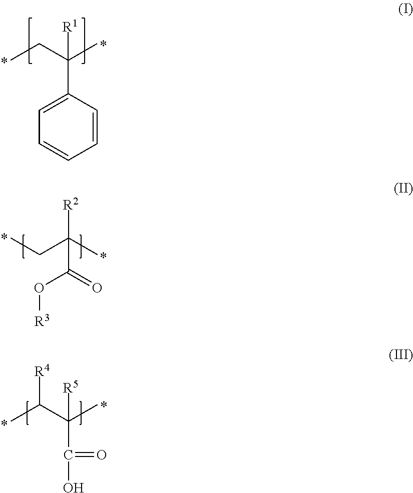

in which

R¹ denotes a hydrogen atom or a methyl group, R² denotes a hydrogen atom or methyl group, R³ denotes a branched (C₁ to C₂₀) alkyl group, a branched (C₂ to C₆) hydroxyalkyl group, or a *-(CH₂CH₂O)ₙ—R⁶ group where R⁶=branched (C₁ to C₂₀) alkyl and n=1 to 30, R⁴ and R⁵ denote a hydrogen atom, or one of the two residues denotes a hydrogen atom and the other a methyl group, and using a delivery apparatus, as a spray onto the keratin-containing fibers.

### DETAILED DESCRIPTION

The following detailed description is merely exemplary in nature and is not intended to limit the invention or the application and uses of the invention. Furthermore, there is no intention to be bound by any theory presented in the preceding background or the following detailed description.

A first subject of the invention is therefore an agent for treating keratin-containing fibers, in particular human hair, containing, in a cosmetically acceptable carrier, at least one anionic setting copolymer encompassing at least one structural unit of formula (I) and at least one structural unit of formula (II) and at least one structural unit of formula (III)

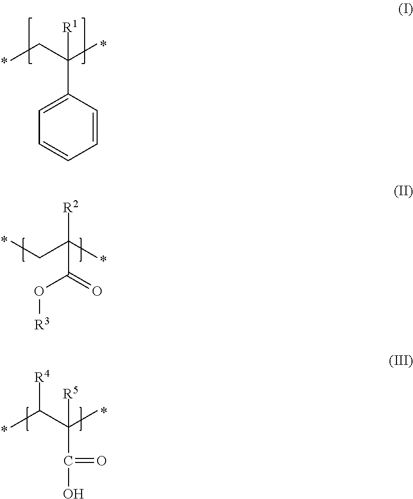

in which
R¹ denotes a hydrogen atom or a methyl group,
R² denotes a hydrogen atom or a methyl group,
R³ denotes a branched (C₁ to C₂₀) alkyl group, a branched (C₂ to C₆) hydroxyalkyl group, or a *-(CH₂CH₂O)ₙ—R⁶ group where R⁶=branched (C₁ to C₂₀) alkyl and n=1 to 30,
R⁴ and R⁵ denote a hydrogen atom, or one of the two residues denotes a hydrogen atom and the other a methyl group.

According to the above formulas and all subsequent formulas, a chemical bond identified with the “*” symbol denotes a “free valence” of the corresponding structural fragment. Further structural fragments bond to this “free valence” in the corresponding copolymer. These are previously described, and optionally additionally further, structural fragments of the respective polymer.

Setting polymers contribute to the hold, and/or to buildup of the hair volume and hair fullness, of the overall hairstyle. These polymers are at the same time also film-forming polymers and are therefore generally typical substances for shape-imparting hair-treatment agents such as hair setting agents, hair foams, hair waxes, hair sprays. It is certainly possible for film formation to be localized, and for only a few fibers to be connected to one another. The so-called “curl retention” test is often used as a test method for the setting effect of a polymer.

“Film-forming polymers” are to be understood as those polymers that, upon drying, leave behind a continuous film on the skin, hair, or nails. Film-formers of this kind can be used in a very wide variety of cosmetic products such as, for example, face masks, make-up, hair setting agents, hair sprays, hair gels, hair waxes, hair therapies, shampoos, or nail polishes. Those polymers which possess sufficient solubility in water or in water/alcohol mixtures to be present in completely dissolved form in the agent according to the present invention are particularly preferred. The film-forming polymers can be of synthetic or natural origin.

“Film-forming polymers” are furthermore understood as those polymers which, when applied in a 0.01- to 20-weight percent (wt %) aqueous, alcoholic, or aqueous alcoholic solution, are capable of depositing a transparent polymer film on the hair.

“Crosslinked” or “crosslinking” is to be understood for purposes of the invention as the linking of polymer chains to one another by covalent chemical bonding, forming a network.

An “anionic polymer” is understood according to the present invention as a polymer that, in a protic solvent under standard conditions, carries structural units having anionic groups that must be compensated for by counterions in order to maintain electroneutrality, and that comprises no structural units having permanently cationic or cationizable groups. “Anionic groups” encompass carboxyl and sulfonic-acid groups.

The anionic setting copolymer encompasses at least one structural unit of formula (I) and at least one structural unit of formula (II) and at least one structural unit of formula (III)

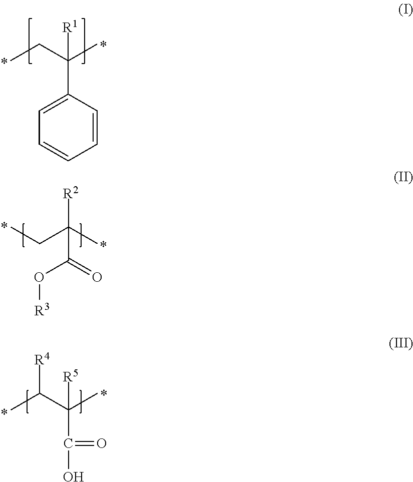

as defined above. In preferred agents according to the present invention, the residue R¹ denotes a hydrogen atom; styrene is therefore a preferred monomer in the copolymers used according to the present invention.

Preferred agents according to the present invention contain as a structural unit (II) esters of acrylic acid or methacrylic acid with branched alcohols; the acrylic acid esters are preferred, so that in preferred agents according to the present invention R² denotes a hydrogen atom.

R³ denotes a branched (C₁ to C₂₀) alkyl group, a branched (C₂ to CO hydroxyalkyl group, or a *-(CH₂CH₂O)ₙ—R⁶ group where R⁶=branched (C₁ to C₂₀) alkyl and n=1 to 30; isopropyl, sec-butyl, tert-butyl, and isooctyl (ethyhexyl) groups are preferred as branched alkyl groups.

It has proven to be advantageous for the hold and shine of hair treated with the agents according to the present invention if the anionic setting copolymers not only comprise a structural unit of formula (II) in which acrylic acid or methacrylic acid is esterified with branched alcohols, but additionally comprise a structural unit in which acrylic acid or methacrylic acid is esterified with unbranched alcohols.

Agents preferred according to the present invention are therefore characterized in that the anionic setting copolymer encompasses at least one structural unit of formula (I) and at least one structural unit of formula (IIa) and at least one structural unit of formula (IIb) and at least one structural unit of formula (III)

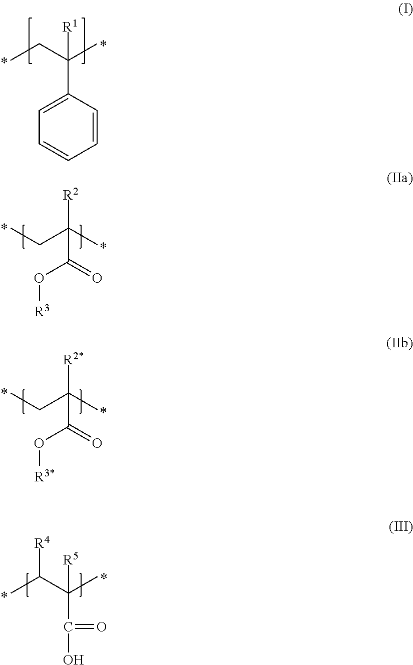

in which
R¹ denotes a hydrogen atom,
R² denotes a hydrogen atom,
R³ denotes an ethylhexyl group,
R²* denotes a hydrogen atom,
R³* denotes a butyl group,
R⁴ and R⁵ denote a hydrogen atom, or one of the two residues denotes a hydrogen atom and the other a methyl group.

As a third obligatory structural unit, the copolymers used in the agents according to the present invention contain acrylic acid (R⁴ and R⁵=—H) or methacrylic acid (R⁴=—CH₃, R⁵=—H).

Very particularly preferred agents according to the present invention contain, as (an) anionic setting polymer(s):
copolymers of styrene with at least isopropyl acrylate and acrylic acid
copolymers of styrene with at least sec-butyl acrylate and acrylic acid
copolymers of styrene with at least tert-butyl acrylate and acrylic acid
copolymers of styrene with at least ethylhexyl acrylate and acrylic acid
copolymers of styrene with at least isopropyl acrylate and methyl acrylate and acrylic acid
copolymers of styrene with at least sec-butyl acrylate and methyl acrylate and acrylic acid
copolymers of styrene with at least tert-butyl acrylate and methyl acrylate and acrylic acid
copolymers of styrene with at least ethylhexyl acrylate and methyl acrylate and acrylic acid
copolymers of styrene with at least isopropyl acrylate and ethyl acrylate and acrylic acid
copolymers of styrene with at least sec-butyl acrylate and ethyl acrylate and acrylic acid
copolymers of styrene with at least tert-butyl acrylate and ethyl acrylate and acrylic acid
copolymers of styrene with at least ethylhexyl acrylate and ethyl acrylate and acrylic acid
copolymers of styrene with at least isopropyl acrylate and propyl acrylate and acrylic acid
copolymers of styrene with at least sec-butyl acrylate and propyl acrylate and acrylic acid
copolymers of styrene with at least tert-butyl acrylate and propyl acrylate and acrylic acid
copolymers of styrene with at least ethylhexyl acrylate and propyl acrylate and acrylic acid
copolymers of styrene with at least isopropyl acrylate and isopropyl acrylate and acrylic acid
copolymers of styrene with at least sec-butyl acrylate and isopropyl acrylate and acrylic acid
copolymers of styrene with at least tert-butyl acrylate and isopropyl acrylate and acrylic acid
copolymers of styrene with at least ethylhexyl acrylate and isopropyl acrylate and acrylic acid
copolymers of styrene with at least isopropyl acrylate and n-butyl acrylate and acrylic acid
copolymers of styrene with at least sec-butyl acrylate and n-butyl acrylate and acrylic acid
copolymers of styrene with at least tert-butyl acrylate and n-butyl acrylate and acrylic acid
copolymers of styrene with at least ethylhexyl acrylate and n-butyl acrylate and acrylic acid
copolymers of styrene with at least isopropyl acrylate and methacrylic acid
copolymers of styrene with at least sec-butyl acrylate and methacrylic acid
copolymers of styrene with at least tert-butyl acrylate and methacrylic acid
copolymers of styrene with at least ethylhexyl acrylate and methacrylic acid
copolymers of styrene with at least isopropyl acrylate and methyl acrylate and methacrylic acid
copolymers of styrene with at least sec-butyl acrylate and methyl acrylate and methacrylic acid
copolymers of styrene with at least tert-butyl acrylate and methyl acrylate and methacrylic acid
copolymers of styrene with at least ethylhexyl acrylate and methyl acrylate and methacrylic acid
copolymers of styrene with at least isopropyl acrylate and ethyl acrylate and methacrylic acid
copolymers of styrene with at least sec-butyl acrylate and ethyl acrylate and methacrylic acid
copolymers of styrene with at least tert-butyl acrylate and ethyl acrylate and methacrylic acid
copolymers of styrene with at least ethylhexyl acrylate and ethyl acrylate and methacrylic acid
copolymers of styrene with at least isopropyl acrylate and propyl acrylate and methacrylic acid
copolymers of styrene with at least sec-butyl acrylate and propyl acrylate and methacrylic acid
copolymers of styrene with at least tert-butyl acrylate and propyl acrylate and methacrylic acid
copolymers of styrene with at least ethylhexyl acrylate and propyl acrylate and methacrylic acid
copolymers of styrene with at least isopropyl acrylate and isopropyl acrylate and methacrylic acid
copolymers of styrene with at least sec-butyl acrylate and isopropyl acrylate and methacrylic acid
copolymers of styrene with at least tert-butyl acrylate and isopropyl acrylate and methacrylic acid
copolymers of styrene with at least ethylhexyl acrylate and isopropyl acrylate and methacrylic acid
copolymers of styrene with at least isopropyl acrylate and n-butyl acrylate and methacrylic acid
copolymers of styrene with at least sec-butyl acrylate and n-butyl acrylate and methacrylic acid
copolymers of styrene with at least tert-butyl acrylate and n-butyl acrylate and methacrylic acid
copolymers of styrene with at least ethylhexyl acrylate and n-butyl acrylate and methacrylic acid.

Agents according to the present invention that are in turn very particularly preferred contain, as (an) anionic setting polymer(s):
copolymers of styrene with isopropyl acrylate and acrylic acid
copolymers of styrene with sec-butyl acrylate and acrylic acid
copolymers of styrene with tert-butyl acrylate and acrylic acid
copolymers of styrene with ethylhexyl acrylate and acrylic acid
copolymers of styrene with isopropyl acrylate and methyl acrylate and acrylic acid
copolymers of styrene with sec-butyl acrylate and methyl acrylate and acrylic acid
copolymers of styrene with tert-butyl acrylate and methyl acrylate and acrylic acid
copolymers of styrene with ethylhexyl acrylate and methyl acrylate and acrylic acid
copolymers of styrene with isopropyl acrylate and ethyl acrylate and acrylic acid
copolymers of styrene with sec-butyl acrylate and ethyl acrylate and acrylic acid
copolymers of styrene with tert-butyl acrylate and ethyl acrylate and acrylic acid
copolymers of styrene with ethylhexyl acrylate and ethyl acrylate and acrylic acid
copolymers of styrene with isopropyl acrylate and propyl acrylate and acrylic acid
copolymers of styrene with sec-butyl acrylate and propyl acrylate and acrylic acid
copolymers of styrene with tert-butyl acrylate and propyl acrylate and acrylic acid
copolymers of styrene with ethylhexyl acrylate and propyl acrylate and acrylic acid
copolymers of styrene with isopropyl acrylate and isopropyl acrylate and acrylic acid
copolymers of styrene with sec-butyl acrylate and isopropyl acrylate and acrylic acid
copolymers of styrene with tert-butyl acrylate and isopropyl acrylate and acrylic acid
copolymers of styrene with ethylhexyl acrylate and isopropyl acrylate and acrylic acid
copolymers of styrene with isopropyl acrylate and n-butyl acrylate and acrylic acid
copolymers of styrene with sec-butyl acrylate and n-butyl acrylate and acrylic acid
copolymers of styrene with tert-butyl acrylate and n-butyl acrylate and acrylic acid
copolymers of styrene with ethylhexyl acrylate and n-butyl acrylate and acrylic acid
copolymers of styrene with isopropyl acrylate and methacrylic acid
copolymers of styrene with sec-butyl acrylate and methacrylic acid
copolymers of styrene with tert-butyl acrylate and methacrylic acid
copolymers of styrene with ethylhexyl acrylate and methacrylic acid
copolymers of styrene with isopropyl acrylate and methyl acrylate and methacrylic acid
copolymers of styrene with sec-butyl acrylate and methyl acrylate and methacrylic acid
copolymers of styrene with tert-butyl acrylate and methyl acrylate and methacrylic acid
copolymers of styrene with ethylhexyl acrylate and methyl acrylate and methacrylic acid
copolymers of styrene with isopropyl acrylate and ethyl acrylate and methacrylic acid
copolymers of styrene with sec-butyl acrylate and ethyl acrylate and methacrylic acid
copolymers of styrene with tert-butyl acrylate and ethyl acrylate and methacrylic acid
copolymers of styrene with ethylhexyl acrylate and ethyl acrylate and methacrylic acid
copolymers of styrene with isopropyl acrylate and propyl acrylate and methacrylic acid
copolymers of styrene with sec-butyl acrylate and propyl acrylate and methacrylic acid
copolymers of styrene with tert-butyl acrylate and propyl acrylate and methacrylic acid
copolymers of styrene with ethylhexyl acrylate and propyl acrylate and methacrylic acid
copolymers of styrene with isopropyl acrylate and isopropyl acrylate and methacrylic acid
copolymers of styrene with sec-butyl acrylate and isopropyl acrylate and methacrylic acid
copolymers of styrene with tert-butyl acrylate and isopropyl acrylate and methacrylic acid
copolymers of styrene with ethylhexyl acrylate and isopropyl acrylate and methacrylic acid
copolymers of styrene with isopropyl acrylate and n-butyl acrylate and methacrylic acid
copolymers of styrene with sec-butyl acrylate and n-butyl acrylate and methacrylic acid
copolymers of styrene with tert-butyl acrylate and n-butyl acrylate and methacrylic acid
copolymers of styrene with ethylhexyl acrylate and n-butyl acrylate and methacrylic acid.

The agent according to the present invention contains the first anionic setting copolymer preferably in a quantity from 0.01 to 29.99 wt %, particularly preferably from 0.1 to 14.9 wt %, very particularly preferably from 0.1 to 9.5 wt %, and most preferably from 0.2 to 7.0 wt %, based in each case on the weight of the total ready-to-use agent.

It has moreover proven to be preferred if the agent according to the present invention contains a total quantity of setting polymers from 0.01 to 30 wt %, particularly preferably from 0.1 to 15 wt %, very particularly preferably from 0.5 to 10.0 wt %, most preferably from 1.0 to 6.0 wt %, based in each case on the weight of the total ready-to-use agent.

It has been possible to increase the effects according to the present invention by the addition of alkyl esters of hydroxy acids, the shine and hold of the hairstyle in particular having been improved. Agents preferred according to the present invention are characterized in that they contain, based in each case on the weight of the total ready-to-use agent, 0.05 to 5 wt %, by preference 0.1 to 2.5 wt %, and in particular 0.15 to 0.5 wt % of at least one alkyl ester of a hydroxy acid.

The (C₂ to C₆) trialkyl citrates are very particularly preferred alkyl esters of hydroxy acids. It is therefore preferred according to the present invention if the preparation additionally contains at least one compound of formula (E)

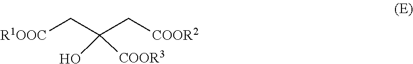

in which
R¹, R², and R³, mutually independently, denote a (C₂ to C₆) alkyl group.

Examples of a (C₂ to C₆) alkyl group according to formula (E) are methyl, ethyl, isopropyl, n-propyl, n-butyl, sec-butyl, isobutyl, tert-butyl, n-pentyl, neopentyl, isopentyl, n-hexyl.

Triethyl citrate has proven to be a particularly preferred compound of formula (E).

The agent according to the present invention contains the compounds of formula (E) preferably in a quantity from 0.01 to 1 wt %, in particular from 0.05 to 0.3 wt %, based in each case on the weight of the total agent. Particularly preferred agents according to the present invention are characterized in that they contain, based in each case on the weight of the total ready-to-use agent, 0.05 to 5 wt %, by preference 0.1 to 2.5 wt %, and in particular 0.15 to 0.5 wt % triethyl citrate.

As already mentioned earlier, the shine and hold of the hairstyle are improved by the additional use of alkyl esters of hydroxy acids. The anionic setting polymers used according to the present invention are synergistically enhanced in terms of their effect in particular by the additional use of alkyl esters of hydroxy acids, an appreciable enhancement in effect being achieved in particular with triethyl citrate.

The agents according to the present invention contain the ingredients or active substances in a cosmetically acceptable carrier.

Preferred cosmetically acceptable carriers are aqueous, alcoholic, or aqueous alcoholic media having by preference at least 10 wt % water, based on the entire agent. The alcohols contained can be, in particular, the lower alcohols having 1 to 4 carbon atoms usually used for cosmetic purposes, for example ethanol and isopropanol.

In the context of a preferred embodiment of the agent according to the present invention, the agent therefore additionally contains at least one alcohol that has 2 to 6 carbon atoms and 1 to 3 hydroxyl groups. This additional alcohol is in turn preferably selected from at least one compound of the group that is constituted from ethanol, ethylene glycol, isopropanol, 1,2-propylene glycol, 1,3-propylene glycol, glycerol, n-butanol, 1,3-butylene glycol. A very particularly preferred alcohol is ethanol.

Very particularly preferred agents according to the present invention are characterized in that they contain, based in each case on the weight of the total ready-to-use agent, 10 to 70 wt %, by preference 20 to 65 wt %, more preferably 30 to 60 wt %, and in particular 35 to 50 wt % ethanol and/or isopropanol.

The additional alcohol having 2 to 6 carbon atoms and 1 to 3 hydroxyl groups is contained in the agent according to the present invention (in particular in the presence of at least one propellant) preferably in a quantity from 40 wt % to 65 wt %, in particular from 40 wt % to 50 wt %, based in each case on the weight of the cosmetic agent.

Organic solvents or a mixture of solvents having a boiling point below 400° C. can be contained as additional co-solvents, in a quantity from 0.1 to 15 weight percent, preferably from 1 to 10 weight percent, based on the total agent. Unbranched or branched hydrocarbons such as pentane, hexane, isopentane, and cyclic hydrocarbons such as cyclopentane and cyclohexane, are particularly suitable as additional co-solvents. Further particularly preferred water-soluble solvents are polyethylene glycol and propylene glycol, in a quantity of up to 30 wt % based on the weight of the entire agent.

The addition in particular of propylene glycol and/or polyethylene glycol and/or polypropylene glycol increases the flexibility of the polymer film formed when the agent according to the present invention is used. If a flexible hold is desired, the agents according to the present invention therefore contain by preference 0.01 to 30 wt % polyethylene glycol and/or polypropylene glycol, based on the total agent.

The agents preferably have a pH from 2 to 11. Particularly preferably, the pH range is between 2 and 8. The indications as to pH refer, for purposes of this document, to the pH at 25° C. unless otherwise noted.

The agents according to the present invention by preference additionally contain at least one surfactant, nonionic, anionic, cationic, and ampholytic surfactants being particularly suitable. The group of the ampholytic or also amphoteric surfactants encompasses zwitterionic surfactants and ampholytes. The surfactants can, according to the present invention, already have an emulsifying effect. The use of at least one nonionic surfactant and/or of at least one cationic surfactant is preferred in the context of this embodiment of the invention.

The additional surfactants are contained in the agent according to the present invention preferably in a quantity from 0.01 wt % to 5 wt %, particularly preferably from 0.05 wt % to 0.5 wt %, based in each case on the weight of the agent.

It has proven to be particularly preferred if the agents according to the present invention additionally contain at least one nonionic surfactant.

Nonionic surfactants contain as a hydrophilic group, for example, a polyol group, a polyalkylene glycol ether group, or a combination of a polyol and polyglycol ether group. Such compounds are, for example:
addition products of 2 to 100 mol ethylene oxide and/or 1 to 5 mol propylene oxide with linear and branched fatty alcohols having 8 to 30 carbon atoms, with fatty acids having 8 to 30 carbon atoms, and with alkylphenols having 8 to 15 carbon atoms in the alkyl group,
addition products, end-capped with a methyl or C₂ to C₆ alkyl residue, of 2 to 50 mol ethylene oxide and/or 1 to 5 mol propylene oxide with linear and branched fatty alcohols having 8 to 30 carbon atoms, with fatty acids having 8 to 30 carbon atoms, and with alkylphenols having 8 to 15 carbon atoms in the alkyl group, such as, for example, the grades obtainable under the marketing designations Dehydrol® LS, Dehydrol® LT (Cognis),
C₁₂ to C₃₀ fatty acid mono- and diesters of addition products of 1 to 30 mol ethylene oxide with glycerol,
addition products of 5 to 60 mol ethylene oxide with castor oil and hardened castor oil,
polyol fatty acid esters such as, for example, the commercial product Hydagen® HSP (Cognis), or Sovermol® grades (Cognis),
alkoxylated triglycerides,
alkoxylated fatty acid alkyl esters of formula (T-I)

R¹CO—(OCH₂CHR²)_{w}OR³ (T-I),
in which R¹CO denotes a linear or branched, saturated and/or unsaturated acyl residue having 6 to 22 carbon atoms, R² denotes hydrogen or methyl, R³ denotes linear or branched alkyl residues having 1 to 4 carbon atoms, and w denotes numbers from 1 to 20,
amine oxides,
hydroxy mixed ethers such as those described, for example, in German Application 19738866,
sorbitan fatty acid esters and addition products of ethylene oxide with sorbitan fatty acid esters, for example the polysorbates,
sugar fatty acid esters and addition products of ethylene oxide with sugar fatty acid esters,
addition products of ethylene oxide with fatty acid alkanolamides and fatty amines,
sugar surfactants of the alkyl and alkenyl oligoglycoside types, in accordance with formula (T-II)

R⁴O-[G]ₚ (T-II),
in which R⁴ denotes an alkyl or alkenyl residue having 4 to 22 carbon atoms, G denotes a sugar residue having 5 or 6 carbon atoms, and p denotes numbers from 1 to 10. They can be obtained in accordance with relevant methods of preparative organic chemistry.

The preferred alkyl and/or alkenyl oligoglycosides are alkyl and/or alkenyl oligoglucosides. The index number p in the general formula (T-II) indicates the degree of oligomerization (DP), i.e. the distribution of mono- and oligoglycosides, and denotes a number between 1 and 10. Whereas p in the individual molecule must always be a whole number, and here can assume especially the values p=1 to 6, the value p for a specific alkyl oligoglycoside is an analytically ascertained calculated value that usually represents a fractional number. Alkyl and/or alkenyl oligoglycosides having an average degree of oligomerization p from 1.1 to 3.0 are preferably used. In terms of applications engineering, those alkyl and/or alkenyl oligoglycosides whose degree of oligomerization is less than 1.7, and in particular between 1.2 and 1.4, are preferred. Alkyl oligoglucosides based on hardened C_{12/14} coconut alcohol having a DP of 1 to 3 are preferred.

The alkylene oxide addition products with saturated linear fatty alcohols and fatty acids having respectively 2 to 100 mol ethylene oxide per mol fatty alcohol or fatty acid have proven to be very particularly preferred nonionic surfactants. Preparations having outstanding properties are likewise obtained when they contain, as nonionic surfactants, C₁₂ to C₃₀ fatty acid mono- and diesters of addition products of 1 to 30 mol ethylene oxide with glycerol and/or addition products of 5 to 60 mol ethylene oxide with castor oil and hardened castor oil.

In the case of the surfactants that represent addition products of ethylene oxide and/or propylene oxide with fatty alcohols, or derivatives of said addition products, both products having a “normal” homolog distribution and those having a restricted homolog distribution can be used. A “normal” homolog distribution is understood as mixtures of homologs that are obtained upon reaction of fatty alcohol and alkylene oxide using alkali metals, alkali metal hydroxides, or alkali metal alcoholates as catalysts. Restricted homolog distributions, on the other hand, are obtained when, for example, hydrotalcites, alkaline-earth metal salts of ethercarboxylic acids, or alkaline-earth metal oxides, hydroxides, or alcoholates are used as catalysts. The use of products having a restricted homolog distribution can be preferred.

Very particularly preferably, the agents according to the present invention contain as a surfactant at least one addition product of 15 to 100 mol ethylene oxide, in particular of 15 to 50 mol ethylene oxide, with a linear or branched (in particular linear) fatty alcohol having 8 to 22 carbon atoms. This refers very particularly preferably to Ceteareth-15, Ceteareth-25, or Ceteareth-50, which are marketed as Eumulgin® CS 15 (COGNIS), Cremophor A25 (BASF SE), or Eumulgin® CS 50 (COGNIS).

All anionic surface-active substances suitable for use on the human body are, in principle, appropriate as anionic surfactants. These are characterized by an anionic group imparting water solubility, for example a carboxylate, sulfate, sulfonate, or phosphate group, and a lipophilic alkyl group having approximately 8 to 30 carbon atoms. Glycol ether or polyglycol ether groups, ester, ether, and amide groups, and hydroxyl groups can additionally be contained in the molecule. Examples of suitable anionic surfactants are, in each case in the form of the sodium, potassium, and ammonium salts and the mono-, di, and trialkanolammonium salts having 2 to 4 carbon atoms in the alkanol group:
linear and branched fatty acids having 8 to 30 carbon atoms (soaps);
ethercarboxylic acids of the formula R—O—(CH₂—CH₂O)ₓ—CH₂—COOH, in which R is a linear alkyl group having 8 to 30 carbon atoms and x=0 or is 1 to 16;
acyl sarcosides having 8 to 24 carbon atoms in the acyl group;
acyl taurides having 8 to 24 carbon atoms in the acyl group;
acyl isethionates having 8 to 24 carbon atoms in the acyl group;
sulfosuccinic acid mono- and dialkyl esters having 8 to 24 carbon atoms in the alkyl group, and sulfosuccinic acid monoalkylpolyoxyethyl esters having 8 to 24 carbon atoms in the alkyl group and 1 to 6 oxyethyl groups;
linear alkanesulfonates having 8 to 24 carbon atoms;
linear alpha-olefinsulfonates having 8 to 24 carbon atoms;
alpha-sulfo fatty acid methyl esters of fatty acids having 8 to 30 carbon atoms;
alkyl sulfates and alkyl polyglycol ether sulfates of the formula R—O(CH₂—CH₂—O)ₓ—OSO₃H, in which R is a preferably linear alkyl group having 8 to 30 carbon atoms and x=0 or is 1 to 12;
mixtures of surface-active hydroxysulfonates;
sulfated hydroxyalkylpolyethylene glycol ethers and/or hydroxyalkylenepropylene glycol ethers;
sulfonates of unsaturated fatty acids having 8 to 24 carbon atoms and 1 to 6 double bonds;
esters of tartaric acid and citric acid with alcohols representing addition products of approximately 2 to 15 molecules of ethylene oxide and/or propylene oxide with fatty alcohols having 8 to 22 carbon atoms;
alkyl and/or alkenyl ether phosphates of formula (T-V)

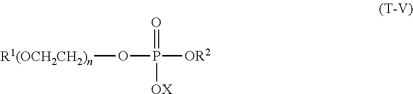

in which R¹ preferably denotes an aliphatic hydrocarbon residue having 8 to 30 carbon atoms, R² denotes hydrogen, a (CH₂CH₂O)ₙR¹ residue, or X, n denotes numbers from 1 to 10, and X denotes hydrogen, an alkali metal or alkaline-earth metal, or NR³R⁴R⁵R⁶ where R³ to R⁶, mutually independently, denote hydrogen or a C₁ to C₄ hydrocarbon residue;
sulfated fatty acid alkylene glycol esters of formula (T-VI)

R⁷CO(AlkO)ₙSO₃M (T-VI)
in which R¹CO denotes a linear or branched, aliphatic, saturated and/or unsaturated acyl residue having 6 to 22 carbon atoms, Alk denotes CH₂CH₂, CHCH₃CH₂, and/or CH₂CHCH₃, n denotes numbers from 0.5 to 5, and M denotes a cation;
amide ethercarboxylic acids;
condensation products of C₈ to C₃₀ fatty alcohols with protein hydrolysates and/or amino acids and derivatives thereof, known to one skilled in the art as protein fatty acid condensates, such as, for example, the Lamepon® grades, Gluadin® grades, Hostapon® KCG, or the Amisoft® grades.

Cationic surfactants of the quaternary ammonium compound, esterquat, and amidoamine types are also usable according to the present invention. Preferred quaternary ammonium compounds are ammonium halides, in particular chlorides and bromides, such as alkyltrimethylammonium chlorides, dialkyldimethylammonium chlorides, and trialkylmethylammonium chlorides. The long alkyl chains of these surfactants preferably have 10 to 18 carbon atoms, for example as in cetyltrimethylammonium chloride, stearyltrimethylammonium chloride, distearyldimethylammonium chloride, lauryldimethylammonium chloride, lauryldimethylbenzylammonium chloride, and tricetylmethylammonium chloride. Further preferred anionic surfactants are the imidazolium compounds known by the INCI names Quaternium-27 and Quaternium-83.

“Zwitterionic surfactants” refers to those surface-active compounds that carry in the molecule at least one quaternary ammonium group and at least one —COO⁽⁻⁾ or SO₃⁽⁻⁾ group. Particularly suitable zwitterionic surfactants are the so-called betaines, such as the N-alkyl-N,N-dimethylammonium glycinates, for example cocalkyldimethylammonium glycinate, N-acylaminopropyl-N,N-dimethylammonium glycinates, for example cocacylaminopropyldimethylammonium glycinate, and 2-alkyl-3-carboxymethyl-3-hydroxyethylimidazolines, having in each case 8 to 18 carbon atoms in the alkyl or acyl group, as well as cocacylaminoethylhydroxyethylcarboxymethyl glycinate. A preferred zwitterionic surfactant is the fatty acid amide derivative known by the INCI name Cocamidopropyl Betaine.

“Ampholytes” are understood to be those surface-active compounds that contain in the molecule, besides a C_{g} to C₂₋₄ alkyl or acyl group, at least one free amino group and at least one —COOH or —SO₃H group, and are capable of forming internal salts. Examples of suitable ampholytic surfactants are N-alkylglycines, N-alkylpropionic acids, N-alkylaminobutyric acids, N-alkyliminodipropionic acids, N-hydroxyethyl-N-alkylamidopropylglycines, N-alkyltaurines, N-alkylsarcosines, 2-alkylaminopropionic acids, and alkylaminoacetic acids, having in each case approximately 8 to 24 carbon atoms in the alkyl group. Particularly preferred ampholytes are N-cocalkylaminopropionate, cocacylaminoethylaminopropionate, and C₁₂ to C₁₈ acyl sarcosine.

The agents according to the present invention can optionally additionally contain at least one film-forming polymer and/or setting polymer. These additional polymers are different from the anionic film-forming copolymers. The optionally added film-forming and/or setting polymers are preferably cationic and/or nonionic.

The cationic film-forming and/or cationic setting polymers can be selected according to the present invention from cationic quaternized cellulose derivatives.

Those cationic quaternized celluloses which carry more than one permanent cationic charge in a side chain generally prove to be advantageous for purposes of the invention.

To be emphasized thereamong are, among the cationic cellulose derivatives, those which are manufactured by the reaction of hydroxyethyl cellulose with a dimethyldiallylammonium reactant (in particular dimethyldiallylammonium chloride), optionally in the presence of further reactants. Particularly suitable in turn among these cationic celluloses are those cationic celluloses having the INCI name Polyquaternium-4, which are marketed e.g. under the names Celquat® H 100, Celquat® L 200 by the National Starch company.

Also suitable are those cationic film-forming and/or cationic setting polymers which encompass at least one structural unit of formula (M-I) and at least one structural unit of formula (M-VI) and optionally at least one structural unit of formula (M-V)

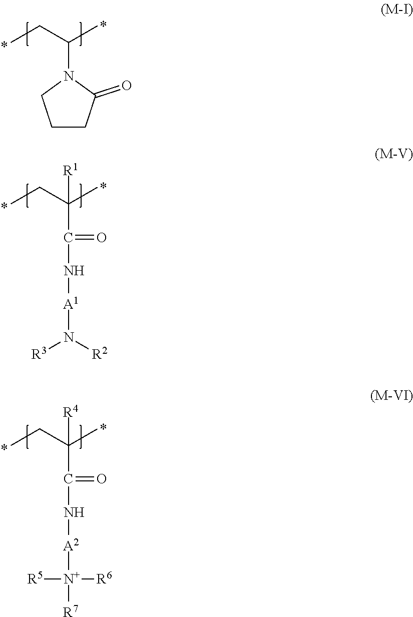

in which
R¹ and R⁴ denote, mutually independently, a hydrogen atom or a methyl group,
A¹ and A² denote, mutually independently, an ethane-1,2-diyl, propane-1,3-diyl, or butane-1,4-diyl group,
R², R³, R⁵, and R⁶ denote, mutually independently, a (C₁ to C₄) alkyl group,
R⁷ denotes a (C₈ to C₃₀) alkyl group.

All possible physiologically acceptable anions, for example chloride, bromide, hydrogen sulfate, methyl sulfate, ethyl sulfate, tetrafluoroborate, phosphate, hydrogen phosphate, dihydrogen phosphate or p-toluenesulfonate, triflate, serve to compensate for the positive charge of monomer (M-VI).

Suitable compounds are commercially obtainable, for example, as
copolymers of dimethylaminoethyl methacrylate, quaternized with diethyl sulfate, with vinylpyrrolidone, having the INCI name Polyquaternium-11, under the designations Gafquat® 440, Gafquat® 734, Gafquat® 755 (each ISP company) and Luviquat PQ 11 PN (BASF SE),
copolymers of methacryloylaminopropyllauryldimethylammonium chloride with vinylpyrrolidone and dimethylaminopropyl methacrylamide, having the INCI name Polyquaternium-55, under the commercial names Styleze® WA O, Styleze® W 20 (ISP company),
copolymers of N-vinylpyrrolidone, N-vinylcaprolactam, N-(3-dimethylaminopropyl) methacrylamide, and 3-(methacryloylamino)propyllauryldimethylammonium chloride (INCI name Polyquaternium-69) under the commercial name Aquastyle® 300 (28 to 32 wt % active substance in ethanol-water mixture) (ISP company).

Further serving as film-forming and/or setting polymers, particularly preferably usable for purposes of the invention, selected from cationic polymers that contain at least one structural unit that comprises a permanently cationized nitrogen atom, are those cationic film-forming and/or cationic setting polymers that comprise at least one structural element of formula (M1)

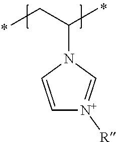

in which
R″ denotes a (C₁ to C₄) alkyl group, in particular a methyl group,
and additionally at least one further cationic and/or nonionic structural element.

All possible physiologically acceptable anions, for example chloride, bromide, hydrogen sulfate, methyl sulfate, ethyl sulfate, tetrafluoroborate, phosphate, hydrogen phosphate, dihydrogen phosphate, or p-toluenesulfonate, triflate, serve to compensate for the positive polymer charge of the components.

It is in turn preferred according to the present invention if at least one copolymer (b1), which encompasses besides at least one structural element of formula (M1) additionally a structural element of formula (M-I)

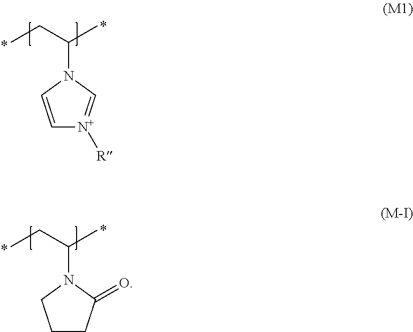

in which
R″ denotes a (C₁ to C₄) alkyl group, in particular a methyl group,
is contained in the agents according to the present invention as a cationic film-forming and/or cationic setting polymer of this embodiment.

All possible physiologically acceptable anions, for example chloride, bromide, hydrogen sulfate, methyl sulfate, ethyl sulfate, tetrafluoroborate, phosphate, hydrogen phosphate, dihydrogen phosphate, or p-toluenesulfonate, triflate, serve to compensate for the positive polymer charge of copolymers (b1).

Very particularly preferred cationic film-forming and/or cationic setting polymers as copolymers (b1) contain 10 to 30 mole percent (mol %), by preference 15 to 25 mol %, and in particular 20 mol % structural units in accordance with formula (M1), and 70 to 90 mol %, by preference 75 to 85 mol % and in particular 80 mol % structural units in accordance with formula (M-I).

It is particularly preferred in this context if copolymers (b1) contain, besides polymer units that result from incorporation of the aforesaid structural units in accordance with formulas (M1) and (M-I) into the copolymer, a maximum of 5 wt %, by preference a maximum of 1 wt %, polymer units that are based on the incorporation of other monomers. Copolymers (b1) are by preference constructed exclusively from structural units of formula (M1), where R″=methyl, and (M-I), and can be described by the general formula (Poly1)

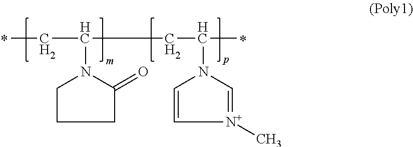

where the indices m and p each vary depending on the molar mass of the polymer and are not intended to signify that these are block copolymers. Structural units of formula (M-1) and of formula (M-I) can instead be present in statistically distributed fashion in the molecule.

If a chloride ion is used to compensate for the positive charge of the polymer of formula (Poly1), these N-methylvinylimidazole/vinylpyrrolidone copolymers are then referred to according to INCI nomenclature as Polyquaternium-16 and are obtainable e.g. from BASF under the commercial names Luviquat® Style, Luviquat® FC 370, Luviquat® FC 550, Luviquat® FC 905, and Luviquat® HM 552.

If a methosulfate is used to compensate for the positive charge of the polymer of formula (Poly1), these N-methylvinylimidazole/vinylpyrrolidone copolymers are then referred to according to INCI nomenclature as Polyquaternium-44 and are obtainable e.g. from BASF under the commercial names Luviquat® UltraCare.

In addition to or instead of the copolymer or copolymers (b1), the agents according to the present invention can also contain copolymers (b2) that, proceeding from copolymer (b1), contain as additional structural units those of formula (M-II)

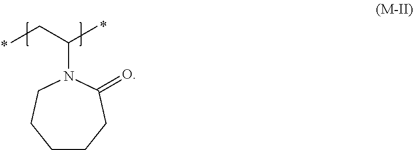

Further particularly preferred agents according to the present invention are thus characterized in that they contain as a cationic setting polymer at least one copolymer (b2) that contains at least one structural unit in accordance with formula (M1-a) and at least a structural unit in accordance with formula (M-I) and at least one structural unit in accordance with formula (M-II)

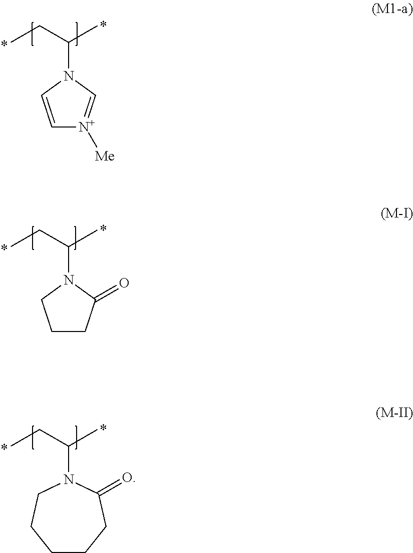

Here as well, it is particularly preferred if copolymers (b2) contain, besides polymer units that result from the incorporation of the aforesaid structural units according to formulas (M1-a), (M-I), and (M-II) into the copolymer, a maximum of 5 wt %, by preference a maximum of 1 wt %, polymer units that are based on the incorporation of other monomers. Copolymers (b2) are by preference constructed exclusively from structural units of formulas (M1-a), (M-I), and (M-II), and can be described by the general formula (Poly2)

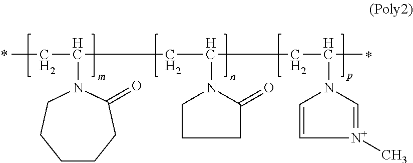

where the indices m, n and p each vary depending on the molar mass of the polymer and are not intended to signify that these are block copolymers. Structural units of the aforesaid formulas can instead be present in statistically distributed fashion in the molecule.

All possible physiologically acceptable anions, for example chloride, bromide, hydrogen sulfate, methyl sulfate, ethyl sulfate, tetrafluoroborate, phosphate, hydrogen phosphate, dihydrogen phosphate or p-toluenesulfonate, triflate, serve to compensate for the positive polymer charge of component (b2).

If a methosulfate is used to compensate for the positive charge of the polymer of formula (Poly2), these N-methylvinylimidazole/vinylpyrrolidone/vinylcaprolactam copolymers are then referred to according to INCI nomenclature as Polyquaternium-46 and are obtainable e.g. from BASF under the commercial name Luviquat® Hold.

Very particularly preferred copolymers (b2) contain 1 to 20 mol %, by preference 5 to 15 mol %, and in particular 10 mol % structural units according to formula (M1-a) and 30 to 50 mol %, by preference 35 to 45 mol %, and in particular 40 mol % structural units according to formula (I) and 40 to 60 mol %, by preference 45 to 55 mol %, and in particular 60 mol % structural units according to formula (M-II).

In addition to or instead of copolymer or copolymers (b1) and/or (b2), the agents according to the present invention can also contain, as a cationic film-forming polymer and/or cationic setting polymer, copolymers (b3) that comprise as structural units those of formulas (M1-a) and (I), as well as further structural units from the group of the vinylimidazole units and further structural units from the group of the acrylamide and/or methacrylamide units.

Further particularly preferred agents according to the present invention are characterized in that they contain, as a cationic film-forming and/or cationic setting polymer, at least one copolymer (b3) that contains at least one structural unit according to formula (M1-a) and at least one structural unit according to formula (M-I) and at least one structural unit according to formula (M-VII) and at least one structural unit according to formula (M-VIII)

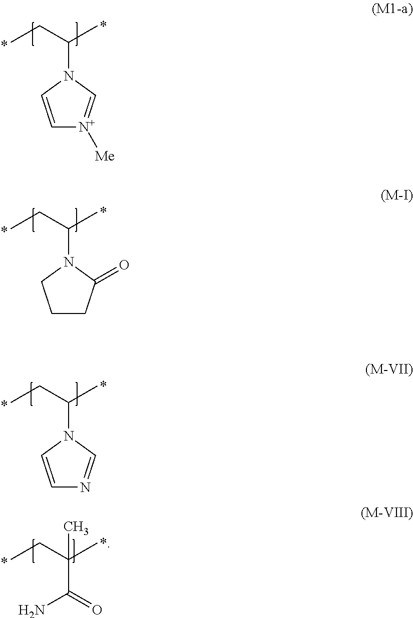

Here as well, it is particularly preferred if copolymers (b3) contain, besides polymer units that result from incorporation of the aforesaid structural units according to formulas (M1-a), (M-I), (M-VII), and (M-VIII) into the copolymer, a maximum of 5 wt %, by preference a maximum of 1 wt %, polymer units that are based on the incorporation of other monomers. Copolymers (b3) are by preference constructed exclusively from structural units of formulas (M1-a), (M-I), (M-VII), and (M-VIII) and can be described by the general formula (Poly3)

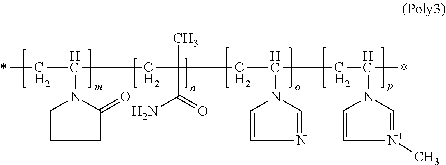

where the indices m, n, o and p each vary depending on the molar mass of the polymer and are not intended to signify that these are block copolymers. Structural units of formulas (M-1a), (M-I), (M-VII), and (M-VIII) can instead be present in statistically distributed fashion in the molecule.

All possible physiologically acceptable anions, for example chloride, bromide, hydrogen sulfate, methyl sulfate, ethyl sulfate, tetrafluoroborate, phosphate, hydrogen phosphate, dihydrogen phosphate or p-toluenesulfonate, triflate, serve to compensate for the positive polymer charge of component (b2).

If a methosulfate is used to compensate for the positive charge of the polymer of formula (Poly3), these N-methylvinylimidazole/vinylpyrrolidone/vinylimidazole/methacrylamide copolymers are referred to according to INCI nomenclature as Polyquaternium-68 and are obtainable e.g. from BASF under the commercial name Luviquat® Supreme.

Very particularly preferred copolymers (b3) contain 1 to 12 mol %, by preference 3 to 9 mol %, and in particular 6 mol % structural units according to formula (M1-a) and 45 to 65 mol %, by preference 50 to 60 mol %, and in particular 55 mol % structural units according to formula (M-I) and 1 to 20 mol %, by preference 5 to 15 mol %, and in particular 10 mol % structural units according to formula (M-VII) and 20 to 40 mol %, by preference 25 to 35 mol %, and in particular 29 mol % structural units according to formula (M-VIII).

Among the additional cationic film-forming and/or setting polymers selected from the cationic polymers having at least one structural element of the above formula (M1), those considered preferred are:
vinylpyrrolidone/1-vinyl-3-methyl-1H-imidazolium chloride copolymers (such as, for example, the one having the INCI name Polyquaternium-16, under the commercial designations Luviquat® Style, Luviquat® FC 370, Luviquat® FC 550, Luviquat® FC 905, and Luviquat® HM 552 (BASF SE)),
vinylpyrrolidone/1-vinyl-3-methyl-1H-imidazolium methyl sulfate copolymers (such as, for example, the one having the INCI name Polyquaternium-44, under the commercial designations Luviquat® Care (BASF SE)),
vinylpyrrolidone/vinylcaprolactam/1-vinyl-3-methyl-1H-imidazolium terpolymers (such as, for example, the one having the INCI name Polyquaternium-46, under the commercial designations Luviquat® Care or Luviquat® Hold (BASF SE)),
vinylpyrrolidone/methacrylamide/vinylimidazole/1-vinyl-3-methyl-1H-imidazolium methyl sulfate copolymers (such as, for example, the one having the INCI name Polyquaternium-68, under the commercial designations Luviquat® Supreme (BASF SE)),
   as well as mixtures of said polymers.

In the context of a preferred embodiment, the agents according to the present invention contain at least one nonionic film-forming and/or nonionic setting polymer as a film-forming and/or setting polymer.

A “nonionic polymer” is understood according to the present invention as a polymer that, in a protic solvent under standard conditions, carries substantially no structural units having permanently cationic or anionic groups that must be compensated for by counterions to maintain electroneutrality. “Cationic groups” encompass, for example, quaternized ammonium groups but not protonated amines. “Anionic groups” encompass, for example, carboxyl and sulfonic-acid groups.

The nonionic film-forming and/or nonionic setting polymers are contained in the agent according to the present invention preferably in a quantity from 0.1 wt % to 20.0 wt %, particularly preferably from 0.2 wt % to 15.0 wt %, very particularly preferably from 0.5 wt % to 5.0 wt %, based in each case on the weight of the agents according to the present invention.

The nonionic film-forming and/or nonionic setting polymers are in turn preferably selected from at least one polymer of the group that is constituted from

homopolymers and nonionic copolymers of N-vinylpyrrolidone,

nonionic copolymers of isobutene,

nonionic copolymers of maleic acid anhydride.

A combination of nonionic film-forming and/or nonionic setting polymers, encompassing at least one nonionic copolymer of maleic acid anhydride and at least one polymer from the group that is constituted from homopolymers and nonionic copolymers of N-vinylpyrrolidone, is preferred.

Suitable polyvinylpyrrolidones are, for example, commercial products such as Luviskol® K 90 or Luviskol® K 85 of the BASF SE company.

Suitable polyvinyl alcohols are marketed, for example, under the commercial designations Elvanol® by Du Pont, or Vinol® 523/540 by the Air Products company.

Suitable polyvinyl acetate is marketed, for example, as an emulsion under the trade name Vinac® by the Air Products company.

Agents that contain, as a nonionic film-forming and/or nonionic setting polymer, at least one polymer selected from the group that is constituted from
copolymer of maleic acid anhydride and methyl vinyl ether,
polyvinylpyrrolidone,
copolymers of N-vinylpyrrolidone and vinyl esters of carboxylic acids having 2 to 18 carbon atoms, in particular of N-vinylpyrrolidone and vinyl acetate,
copolymers of N-vinylpyrrolidone and N-vinylimidazole and methacrylamide,
copolymers of N-vinylpyrrolidone and N-vinylimidazole and acrylamide,
copolymers of N-vinylpyrrolidone with N,N-di(C₁ to C₄) alkylamino-(C₂ to C₄) alkylacrylamide,
copolymers of N-vinylpyrrolidone with N,N-di(C₁ to C₄) alkylamino-(C₂ to C₄) alkylacrylamide,
   or mixtures of these polymers, are very particularly preferred according to the present invention.

It is in turn preferred if the molar ratio between the structural units from the N-vinylpyrrolidone monomer and the structural units from the vinyl acetate monomer that are contained in the polymer is in the range from 20 to 80 to 80 to 20, in particular from 30 to 70 to 60 to 40.

Suitable copolymers of vinylpyrrolidone and vinyl acetate are obtainable, for example, under the trademark Luviskol® VA 37, Luviskol® VA 55, Luviskol® VA 64, and Luviskol® VA 73 from the BASF SE company.

Further preferred agents according to the present invention of this embodiment are characterized in that they contain, as a nonionic film-forming and/or nonionic setting polymer, at least one copolymer (n1) that contains at least one structural unit according to formula (M-I) and at least one structural unit according to formula (M-VII) and at least one structural unit according to formula (M-VIII)

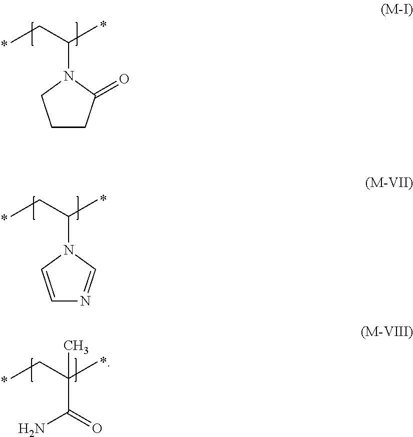

Here as well, it is particularly preferred if these copolymers contain, besides polymer units that result from incorporation of the aforesaid structural units according to formulas (M1-a), (I), (VII), and (VIII) into the copolymer, a maximum of 5 wt %, by preference a maximum of 1 wt %, polymer units that are based on the incorporation of other monomers. Copolymers (n1) are by preference constructed exclusively from structural units of formulas (M1-a), (I), (VII), and (VIII) and can be described by the general formula (Poly4)

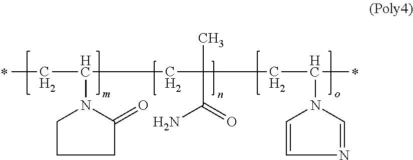

where the indices m, n, o and p each vary depending on the molar mass of the polymer and are not intended to signify that these are block copolymers. Structural units of formulas (I), (VII), and (VIII) can instead be present in statistically distributed fashion in the molecule.

A particularly preferred polymer is selected in this context from the polymers having the INCI name VP/Methacrylamide/Vinyl Imidazole Copolymer, which are obtainable e.g. under the trade name Luviset Clear from the BASF SE company.

Also suitable according to the present invention are those agents that contain at least one nonionic film-forming and/or nonionic setting polymer encompassing at least one structural unit of formula (M-I) and at least one structural unit of formula (M-III)

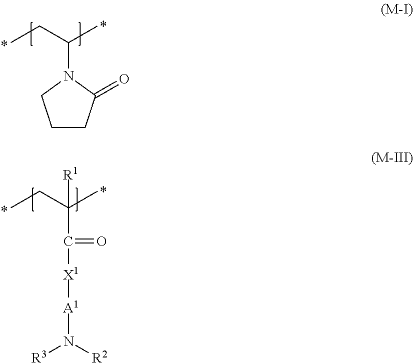

in which
R¹ denotes a hydrogen atom or a methyl group,
X¹ denotes an oxygen atom or an NH group,
A¹ denotes an ethane-1,2-diyl, propane-1,3-diyl, or butane-1,4-diyl group,
R² and R³ denote, mutually independently, a (C₁ to C₄) alkyl group.

It is particularly preferred in this context if the above nonionic film-forming and/or nonionic setting polymer is selected from at least one polymer that meets one or more of the following criteria:
R¹ signifies a methyl group,
X¹ denotes an NH group,
A¹ denotes ethane-1,2-diyl or propane-1,3-diyl,
R² and R³ denote, mutually independently, methyl or ethyl (particularly preferably methyl).

Particularly preferably, the nonionic film-forming and/or nonionic setting polymers of this embodiment are at least one polymer that encompasses at least one structural unit of formula (M-I) and at least one structural unit of formula (M-III-8)

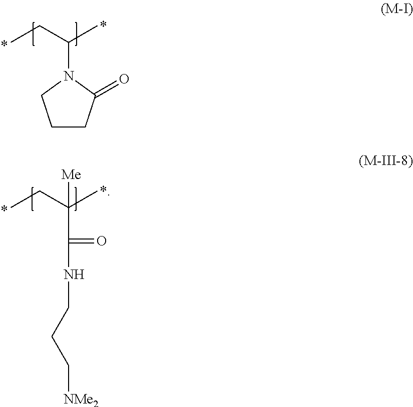

A very particularly preferred nonionic film-forming and/or nonionic setting polymer of this embodiment is a copolymer of N-vinylpyrrolidone and N,N-dimethylaminopropyl methacrylamide that is sold, for example, with the INCI name VP/DMAPA Acrylates Copolymer, for example under the trade name Styleze® CC-10, by the ISP company.

The agents according to the present invention can furthermore contain the adjuvants and additives that are usually added to conventional styling agents.

Additional care-providing substances are to be recited in particular as suitable adjuvants and additives.

The agent can contain as a care-providing substance, for example, at least one protein hydrolysate and/or one of its derivatives.

Protein hydrolysates are product mixtures obtained by the acid-, base-, or enzyme-catalyzed breakdown of proteins. The term “protein hydrolysates” is also understood according to the present invention to mean total hydrolysates as well as individual amino acids and derivatives thereof, as well as mixtures of different amino acids. Polymers constructed from amino acids and amino acid derivatives are also understood according to the present invention under the term “protein hydrolysates.” Beta-amino acids and derivatives thereof can of course also be used according to the present invention. The molecular weight of the protein hydrolysates usable according to the present invention is between 75 (the molecular weight of glycine) and 200,000 Daltons (D); the molecular weight is equal to preferably 75 to 50,000 D, and very particularly preferably to 75 to 20,000 D.

According to the present invention, protein hydrolysates of both vegetable and animal origin, or of marine or synthetic origin, can be used.

Although the use of protein hydrolysates as such is preferred, amino acid mixtures obtained in other ways can also optionally be used instead of them.

The protein hydrolysates can be contained in the agents according to the present invention, for example, in concentrations from 0.01 wt % to 20 wt %, by preference from 0.05 wt % to 15 wt %, and very particularly preferably in quantities from 0.05 wt % to 5 wt %, based in each case on the total application preparation.

The agent according to the present invention can further contain at least one vitamin, provitamin, vitamin precursor, and/or one of their derivatives as a care-providing substance.

Those vitamins, provitamins, and vitamin precursors which are usually assigned to groups A, B, C, E, F, and H are preferred according to the present invention.

The agents according to the present invention preferably contain vitamins, provitamins, and vitamin precursors from groups A, B, C, E and H. Panthenol, pantolactone, pyridoxine and derivatives thereof, as well as nicotinic acid amide and biotin, are particularly preferred.

Like the addition of glycerol and/or propylene glycol, the addition of panthenol increases the flexibility of the polymer film formed upon application of the agent according to the present invention. If a particularly flexible hold is desired, the agents according to the present invention can thus contain panthenol instead of or in addition to glycerol and/or propylene glycol. In a preferred embodiment the agents according to the present invention contain panthenol, by preference in a quantity from 0.05 to 10 wt %, particularly preferably 0.1 to 5 wt %, based in each case on the total agent.

The agents according to the present invention can further contain at least one plant extract as a care-providing substance. These extracts are usually produced by extraction of the entire plant. In individual cases, however, it may also be preferred to produce the extracts exclusively from blossoms and/or from leaves of the plant. According to the present invention the extracts from green tea, oak bark, nettle, hamamelis, hops, henna, chamomile, burdock root, horsetail, hawthorn, linden blossoms, almond, aloe vera, pine needles, horse chestnut, sandalwood, juniper, coconut, mango, apricot, lemon, wheat, kiwi fruit, melon, orange, grapefruit, salvia, rosemary, birch, mallow, lady's-smock, wild thyme, yarrow, thyme, lemon balm, restharrow, coltsfoot, hibiscus, meristem, ginseng, and ginger root are especially preferred.

The plant extracts can be used according to the present invention in both pure and diluted form. If they are used in diluted form, they usually contain approximately 2 to 80 wt % active substance and, as a solvent, the extraction agent or extraction agent mixture used to recover them. It can further be preferred to use in the agents according to the present invention mixtures of several, in particular two, different plant extracts. The agents according to the present invention contain these care-providing substances preferably in quantities from 0.001 to 2, in particular from 0.01 to 0.5 wt %, based in each case on the total application preparation.

It has been found that the addition of a UV filter allows both the agents themselves, and the treated fibers, to be protected from damaging influences of UV radiation. Surprisingly, the shine of the fibers treated with the agents according to the present invention also becomes improved and more long-lasting. At least one UV filter is therefore preferably added to the agent. The UV filters are contained usually in quantities from 0.01 to 5 wt %, based on the total application preparation. Quantities from 0.1 to 2.5 wt % are preferred. Agents preferred according to the present invention contained, based in each case on the weight of the total ready-to-use agent, 0.05 to 5 wt %, by preference 0.1 to 2.5 wt %, and in particular 0.15 to 0.5 wt % UV filter substances.

The suitable UV filters are not subject to any general restrictions in terms of their structure and their physical properties. Instead, all UV filters usable in the cosmetics sector, whose absorption maximum lies in the UVA (315 to 400 nm) UVB (280 to 315 nm), or UVC (<280 nm) regions, are suitable. UV filters having an absorption maximum in the UVB region, in particular in the region from approximately 280 to approximately 300 nm, are particularly preferred.

The UV filters preferred according to the present invention can be selected, for example, from substituted benzophenones, p-aminobenzoic acid esters, diphenylacrylic acid esters, cinnamic acid esters, salicylic acid esters, benzimidazoles, cinnamates, and o-aminobenzoic acid esters.

Those UV filters whose molar extinction coefficient at the absorption maximum is above 15,000, in particular above 20,000, are preferred.

Very particularly preferred agents according to the present invention contain, based in each case on the weight of the total ready-to-use agent, 0.05 to 5 wt %, by preference 0.1 to 2.5 wt %, and in particular 0.15 to 0.5 wt % benzophenone-4 and/or isoamyl p-methoxycinnamate.

Besides the components recited, the agents can furthermore contain all active substances, additives, and adjuvants known for such preparations.

Further active substances, additives, and adjuvants are, for example
thickening agents such as agar-agar, guar gum, alginates, xanthan gum, gum arabic, karaya gum, locust bean flour, linseed gums, dextrans, cellulose derivatives, e.g. methyl cellulose, hydroxyalkyl cellulose, and carboxymethyl cellulose, starch fractions and derivatives such as amylose, amylopectin, and dextrins, clays such as e.g. bentonite, or entirely synthetic hydrocolloids such as, for example, polyvinyl alcohol, and optionally crosslinked polyacrylates,
structuring agents such as maleic acid and lactic acid,
perfume oils, dimethylisosorbide, and cyclodextrins,
defoamers such as silicones,
dyes for coloring the agent,
anti-dandruff active substances such as piroctone olamide, zinc omadine, and climbazol,
substances for adjusting pH, such as e.g. usual acids, in particular edible acids, and bases,
consistency agents such as sugar esters, polyol esters, or polyolalkyl ethers,
complexing agents such as EDTA, NTA, β-alaninediacetic acid, and phosphonic acids,
swelling and penetration substances such as glycerol, propylene glycol monoethyl ether, carbonates, hydrogen carbonates, guanidines, ureas, and primary, secondary, and tertiary phosphates,
opacifiers such as latex, styrene/PVP and styrene/acrylamide copolymers,
luster agents such as ethylene glycol mono- and distearate as well as PEG-3 distearate,
preservatives,
antioxidants.

With regard to further optional components as well as the quantities of those components used, reference is made expressly to the relevant manuals known to one skilled in the art.

The agents according to the present invention can be formulated in all forms usual for styling agents, for example in the form of solutions that can be applied onto the hair as a lotion or as a pump spray or aerosol spray, or of other preparations that are suitable for use on the hair.

The agents according to the present invention are configured by preference as a pump spray, aerosol spray, pump foam, or aerosol foam. For this, the agents according to the present invention are packaged in a delivery apparatus that is either a pressurized-gas container additionally filled with a propellant (“aerosol container”) or a non-aerosol container. The pressurized-gas containers with which a product is distributed through a valve as a result of the internal gas pressure of the container are referred to by definition as “aerosol containers.” A “non-aerosol container” is defined, conversely to the “aerosol” definition, as a vessel under standard pressure with which a product is distributed by means of mechanical action by way of a pump system or squeeze system.

The agents according to the present invention are present particularly preferably as an aerosol hair foam or aerosol hair spray. The agent according to the present invention therefore preferably additionally contains at least one propellant.

Agents according to the present invention that are present in the form of an aerosol product can be manufactured in usual fashion. As a rule all the constituents of the agent according to the present invention, with the exception of the propellant, are introduced into a suitable pressure-tight container. The latter is then sealed with a valve. Lastly, the desired quantity of propellant is introduced using conventional techniques. A further subject of the present invention is therefore a kit of parts encompassing an aerosol delivery apparatus and a composition that contains, based on its weight:
30 to 80 wt %, by preference 40 to 70 wt %, and in particular 50 to 60 wt % of an agent according to the present invention, and
20 to 70 wt %, by preference 30 to 60 wt %, and in particular 40 to 50 wt % of at least one propellant.

In the embodiment as an aerosol spray, propellants suitable according to the present invention are selected, for example, from N₂O, dimethyl ether, CO₂, air, alkanes having 3 to 5 carbon atoms such as propane, n-butane, isobutane, n-pentane, and isopentane, and mixtures thereof. Dimethyl ether, propane, n-butane, isobutane, and mixtures thereof are preferred. In accordance with a preferred embodiment, the aforesaid alkanes, mixtures of the aforesaid alkanes, or mixtures of the aforesaid alkanes with dimethyl ether are used as the only propellant. The invention also expressly encompasses, however, the concurrent use of propellants of the fluorochlorocarbon type, but in particular the fluorocarbons.

The propellant is contained in the agents according to the present invention, in the embodiment as an aerosol spray, preferably in a quantity from 30 to 60 wt %, based on the weight of the total agent.

Very particularly preferably, mixtures of propane and butane are used as the only propellant, at a weight ratio of propane to butane from 20 to 80 to 15 to 85. These mixtures are in turn used in the agents according to the present invention preferably in a quantity from 30 to 55 wt %, based on the weight of the total agent. “Butane” is understood according to the present invention as n-butane, isobutane, and mixtures of n-butane and isobutane.

For a given spray apparatus, the sizes of the aerosol droplets and the respective size distribution can be adjusted by way of the quantitative ratio of propellant to the other constituents of the preparations.

The spray rate of the sprays according to the present invention is preferably 6.5 to 10.0 g/10 s.

Particularly preferred agents according to the present invention (aerosol sprays) are packaged in an aerosol container having a stem valve with a stem orifice 0.27 to 0.35 mm in diameter. Such valves are marketed, for example, by the Coster company as valves of the KE or KEN type.

In the embodiment as an aerosol foam, propellants suitable according to the present invention are selected, for example, from N₂O, dimethyl ether, CO₂, air, alkanes having 3 to 5 carbon atoms such as propane, n-butane, isobutane, n-pentane, and isopentane, and mixtures thereof. The invention also expressly encompasses, however, the concurrent use of propellants of the fluorochlorocarbon type, but in particular the fluorocarbons.

In accordance with the embodiment of an aerosol foam, the aforesaid alkanes, mixtures of the aforesaid alkanes, or mixtures of the aforesaid alkanes with dimethyl ether are preferably used as the only propellant. Particularly preferred propellants are dimethyl ether, propane, n-butane, isobutane, and mixtures thereof.

For a given spray apparatus, the sizes of the foam bubbles and the respective size distribution can be adjusted by way of the quantitative ratio of propellant to the other constituents of the preparations.

When conventional aerosol containers are used, aerosol foam products contain the propellant preferably in quantities from 1 to 35 wt %, based on the total product. Quantities from 2 to 30 wt %, in particular from 3 to 15 wt %, are particularly preferred.

Isopentane is preferably suitable as a propellant for foaming gel-type agents in a two-chamber aerosol container, said propellant being incorporated into the agents according to the present invention and being packaged in the first chamber of the two-chamber aerosol container. Packaged in the second chamber of the two-chamber aerosol container is at least one further propellant, different from isopentane, that builds up in the two-chamber aerosol container a higher pressure than the isopentane. The propellants of the second chamber are preferably selected from N₂O, dimethyl ether, CO₂, air, alkanes having 3 or 4 carbon atoms (such as propane, n-butane, isobutane), as well as mixtures thereof.

The use of the aforementioned additional preferred ingredients, and of the utilization quantities or utilization quantity ratios characterized as preferred, is of course also preferred in the context of all aerosol foam embodiment(s).

The agents according to the present invention, and products that contain the agents according to the present invention, in particular aerosol hair sprays and aerosol hair foams, are notable in particular for the fact that they impart to treated hair a very natural shine and a strong hold. A second subject of the invention is the use of an agent of the first subject of the invention in order to produce shine on keratin-containing fibers, in particular on human hair.

The products according to the present invention that contain these agents, in particular aerosol hair sprays, are notable in particular for the fact that they impart to treated hair a very strong, lasting hairstyle hold even though the hair remains flexible. A third subject of the invention is therefore the use of an agent of the first subject of the invention for the temporary reshaping of keratin-containing fibers, in particular human hair.

A further subject of the invention is a method for treating keratin-containing fibers, in particular human hair, in which an agent according to the first subject of the invention is applied, using a delivery apparatus, as a spray onto the keratin-containing fibers.

It is preferred according to the present invention in this context that the keratin-containing fibers be shaped, and that that shape be fixed using the agent of the first subject of the invention.

It is further preferred if, after application of the agent according to the present invention, the agent according to the present invention remains on the keratin-containing fibers, i.e., is not rinsed out again. The delivery apparatuses or aerosol products recited previously are considered to be preferred according to the present invention.

A further subject of the invention is a method for treating keratin-containing fibers, in particular human hair, in which method, using a delivery apparatus, an agent according to the first subject of the invention is foamed into a foam and the resulting foam is applied onto the keratin-containing fibers.

It is preferred according to the present invention in this context that the keratin-containing fibers be shaped, and that that shape be fixed using the agent of the first subject of the invention.

It is further preferred if, after application of the agent according to the present invention, the agent according to the present invention remains on the keratin-containing fibers, i.e. is not rinsed out again. The delivery apparatuses recited previously are considered to be preferred according to the present invention.

### EXAMPLES

The following formulations were prepared by mixing the raw materials indicated, and were decanted into an aerosol can having a model KE valve of the Coster company and a model V06 “Standard micromist insert” swirl nozzle of the Coster company. The aerosol cans were sealed with the valve, and lastly the corresponding propellant (here dimethyl ether) was added. The following quantitative indications are understood, unless otherwise noted, as percentages by weight.

TABLE 1
Formulations

| Raw materials | E1 | E2 | E3 | E4 | E5 | E6 | E7 | E8 |
|---|---|---|---|---|---|---|---|---|
| Aminomethyl- | 0.95 | 0.98 | 1.01 | 1.04 | 1.06 | 1.09 | 1.12 | 1.18 |
| propanol | | | | | | | | |
| Anionic setting | 8.5 | 8.75 | 9.0 | 9.25 | 9.5 | 9.75 | 10.0 | 10.5 |
| polymer¹ | | | | | | | | |
| Triethyl citrate | 0.2 | 0.2 | 0.2 | 0.2 | 0.25 | 0.25 | 0.25 | 0.25 |
| Benzophe- | 0.15 | 0.15 | 0.2 | 0.2 | 0.2 | 0.2 | 0.25 | 0.25 |
| none-4 | | | | | | | | |
| Perfume | 0.2 | 0.2 | 0.2 | 0.2 | 0.2 | 0.2 | 0.2 | 0.2 |
| Dimethyl ether | 40.00 | 40.00 | 40.00 | 40.00 | 50.00 | 50.00 | 50.00 | 50.00 |

| | |
|---|---|
| Ethanol | to 100 |

TABLE 2

| Raw materials | E9 | E10 | E11 | E12 | E13 | E14 | E15 | E16 |
|---|---|---|---|---|---|---|---|---|
| Aminomethyl- | 0.72 | 0.75 | 0.78 | 0.81 | 0.84 | 0.87 | 0.90 | 0.95 |
| propanol | | | | | | | | |
| Anionic setting | 8.5 | 8.75 | 9.0 | 9.25 | 9.5 | 9.75 | 10.0 | 10.5 |
| polymer¹ | | | | | | | | |
| Triethyl citrate | 0.2 | 0.2 | 0.2 | 0.2 | 0.25 | 0.25 | 0.25 | 0.25 |
| Benzophe- | 0.15 | 0.15 | 0.2 | 0.2 | 0.2 | 0.2 | 0.25 | 0.25 |
| none-4 | | | | | | | | |
| Perfume | 0.2 | 0.2 | 0.2 | 0.2 | 0.2 | 0.2 | 0.2 | 0.2 |
| Dimethyl ether | 40.00 | 40.00 | 40.00 | 40.00 | 50.00 | 50.00 | 50.00 | 50.00 |

| | |
|---|---|
| Ethanol | to 100 |

¹Copolymer encompassing structural units derived from styrene, methacrylic acid, ethylhexyl acrylate, and butyl acrylate (40% active substance)

After application of each of the agents E1 to E16 onto a strand of hair, these agents according to the present invention produced an outstanding hairstyle hold. The hair acquired a natural shine and had a supple feel.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. An agent for treating keratin-containing fibers, comprising, in a cosmetically acceptable carrier:
at least one anionic setting copolymer comprising at least one structural unit of formula (I) and at least one structural unit of formula (II) and at least one structural unit of formula (III) 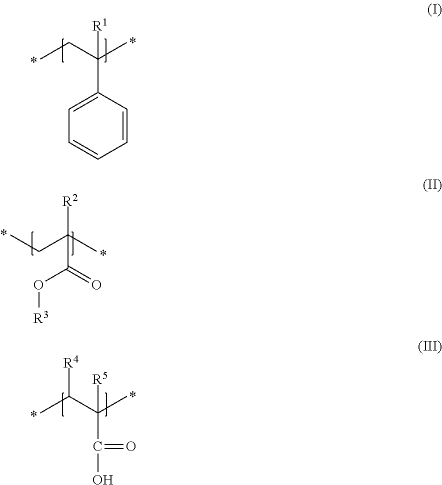
in which
R¹ denotes a hydrogen atom or a methyl group,
R² denotes a hydrogen atom or a methyl group,
R³ denotes a branched (C₁ to C₂₀) alkyl group, a branched (C₂ to C₆) hydroxyalkyl group, or a *-(CH₂CH₂O)ₙ—R⁶ group where R⁶=branched (C₁ to C₂₀) alkyl and n=1 to 30,
R⁴ and R⁵ denote a hydrogen atom, or one of the two residues denotes a hydrogen atom and the other a methyl group.

2. The agent according to claim 1, wherein the anionic setting copolymer comprises at least one structural unit of formula (I) and at least one structural unit of formula (IIa) and at least one structural unit of formula (IIb) and at least one structural unit of formula (III) 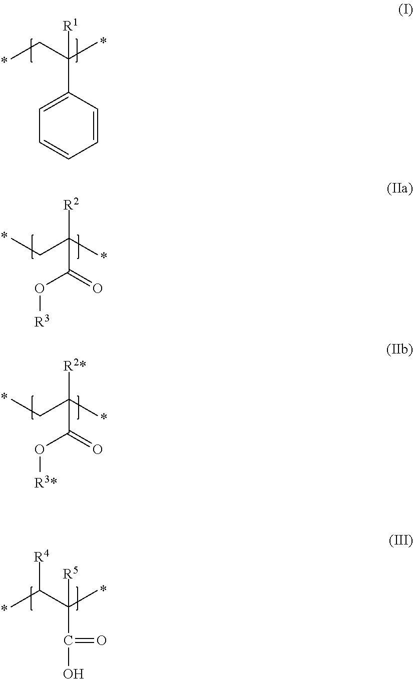
in which
R¹ denotes a hydrogen atom,
R² denotes a hydrogen atom,
R³ denotes an ethylhexyl group,
R²* denotes a hydrogen atom,
R³* denotes a butyl group,
R⁴ and R⁵ denote a hydrogen atom, or one of the two residues denotes a hydrogen atom and the other a methyl group.

3. The agent according to claim 1, wherein the anionic setting copolymer is contained in a quantity from 0.01 to 29.99 wt %, based in each case on the weight of the total ready-to-use agent.

4. The agent according to claim 1, comprising, based in each case on the weight of the total ready-to-use agent, 0.05 to 5 wt % of at least one alkyl ester of a hydroxy acid.

5. The agent according to claim 4, comprising, based in each case on the weight of the total ready-to-use agent, 0.05 to 5 wt % of triethyl citrate.

6. The agent according to claim 1, comprising, based in each case on the weight of the total ready-to-use agent, 10 to 70 wt % ethanol and/or isopropanol.

7. The agent according to claim 1, comprising, based in each case on the weight of the total ready-to-use agent, 0.05 to 5 wt % of UV filter substances.

8. The agent according to claim 7, comprising, based in each case on the weight of the total ready-to-use agent, 0.05 to 5 wt % benzophenone-4 and/or isoamyl p-methoxycinnamate.

9. A kit of parts comprising an aerosol delivery apparatus and a composition comprising, based on its weight:
30 to 80 wt % of an agent for treating keratin-containing fibers, comprising, in a cosmetically acceptable carrier: at least one anionic setting copolymer comprising at least one structural unit of formula (I) and at least one structural unit of formula (II) and at least one structural unit of formula (III) 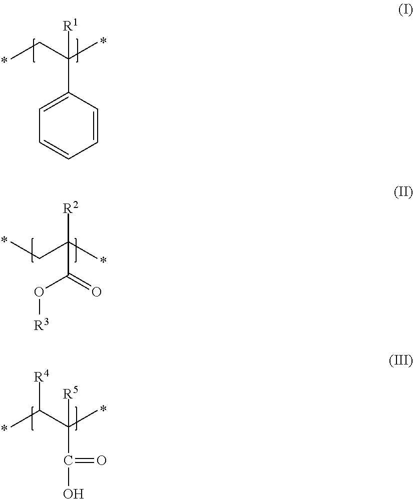
in which
R¹ denotes a hydrogen atom or a methyl group, R² denotes a hydrogen atom or methyl group, R³ denotes a branched (C₁ to C₂₀) alkyl group, a branched (C₂ to C₆) hydroxyalkyl group, or a *-(CH₂CH₂O)ₙ—R⁶ group where R⁶=branched (C₁ to C₂₀) alkyl and n=1 to 30, R⁴ and R⁵ denote a hydrogen atom, or one of the two residues denotes a hydrogen atom and the other a methyl group, and
20 to 70 wt % of at least one propellant.

10. A method for treating keratin-containing fibers comprising, applying an agent for treating keratin-containing fibers, comprising, in a cosmetically acceptable carrier: at least one anionic setting copolymer comprising at least one structural unit of formula (I) and at least one structural unit of formula (II) and at least one structural unit of formula (III) 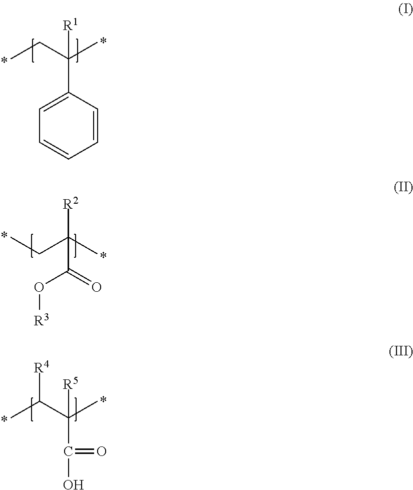
in which
R¹ denotes a hydrogen atom or a methyl group, R² denotes a hydrogen atom or methyl group, R³ denotes a branched (C₁ to C₂₀) alkyl group, a branched (C₂ to C₆) hydroxyalkyl group, or a *-(CH₂CH₂O)ₙ—R⁶ group where R⁶=branched (C₁ to C₂₀) alkyl and n=1 to 30, R⁴ and R⁵ denote a hydrogen atom, or one of the two residues denotes a hydrogen atom and the other a methyl group, and
using a delivery apparatus, as a spray onto the keratin-containing fibers.
